(19) United States
(12) Patent Application Publication
(10) Pub. No.: US 2020/0000847 A1
(43) Pub. Date: Jan. 2, 2020

(54) PHARMACEUTICAL COMPOSITION FOR TREATING LEUKEMIA AND METHOD FOR PREPARING THE SAME

(71) Applicants: Yifan Pharmaceutical Academy (Beijing) Co., Ltd., Beijing (CN); Yifan Pharmaceutical Co., Ltd., Hangzhou (CN)

(72) Inventors: Xianfeng CHENG, Hangzhou (CN); Wenyan ZHOU, Hangzhou (CN); Xiaodong LIANG, Beijing (CN); Liping QIAN, Hangzhou (CN)

(21) Appl. No.: 16/563,996

(22) Filed: Sep. 9, 2019

Related U.S. Application Data
(63) Continuation-in-part of application No. 15/781,213, filed on Jun. 4, 2018.

(30) Foreign Application Priority Data
Dec. 17, 2015 (CN) 201510955934.5

(51) Int. Cl. A61K 33/36; A61K 36/195; A61K 36/537; A61K 36/13; A61K 9/20; A61P 35/02

(52) U.S. Cl. CPC A61K 33/36; A61K 36/195; A61P 35/02; A61K 36/13; A61K 9/2095; A61K 36/537

(57) ABSTRACT

A pharmaceutical composition for treating leukemia, including by weight 2 to 8% of realgar, 25 to 42% of indigo naturalis, 50 to 60% of salvia miltiorrhiza, and 6 to 10% of heterophylla.

### CROSS-REFERENCE TO RELATED APPLICATIONS

This application is a continuation-in-part of and claims domestic priority benefits to U.S. application Ser. No. 15/781,213, filed on Jun. 4, 2018, now pending, which is a national phase entry of International Application No. PCT/CN2016/078911 with an international filing date of Apr. 8, 2016, designating the United States, and further claims priority benefits to Chinese Patent Application No. 201510955934.5 filed Dec. 17, 2015. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, and Cambridge, Mass. 02142.

### BACKGROUND OF THE INVENTION

### Field of the Invention

The invention relates to the field of drugs for treating leukemia, in particular to a pharmaceutical composition for treating leukemia and a method for preparing the same.

### Description of the Related Art

Long-term administration of traditional Chinese medicines containing excess realgar causes chronic arsenic poisoning or even death. Chinese Pharmacopoeia, 2010 version, states that a daily intake of realgar must not exceed 0.1 g. If realgar is administered in large doses continuously, arsenic would accumulate in the body, causing damage to liver, kidney, circulatory system, and nervous system.

Chinese Patent Application No. 200410050453.1 discloses an anti-leukemia preparation of traditional Chinese medicine containing realgar. However, conventional methods of making this preparation are not efficient, and a simultaneous use of this preparation and retinoic acid causes cross-resistance.

The main component of realgar is arsenic disulfide or tetraarsenic tetrasulfide. The main impurity in realgar is arsenic trioxide. Due to the toxicity of conventional realgar tablets, their long-term use may result in damage to the liver and kidneys.

Salvia miltiorrhiza exhibits various pharmacological effects, notably it reduces myocardial ischemia, cerebral ischemia, and thrombosis by improving microcirculation, and promoting tissue repair and regeneration. It exhibits sedation and analgesia. However, tanshinone and tanshinol, which are the active ingredients of salvia miltiorrhiza, are unstable and decompose at high temperature.

### SUMMARY OF THE INVENTION

In view of the above-described problems, it is one objective of the present disclosure to provide a pharmaceutical composition for treatment of leukemia.

It is another objective of the present disclosure to provide a method for preparing a pharmaceutical composition for treatment of leukemia.

In one embodiment, provided is a pharmaceutical composition for treating leukemia, comprising by weight 2 to 8% of realgar, 25 to 42% of indigo naturalis, 50 to 60% of salvia miltiorrhiza, and 6 to 10% of heterophylla.

In a class of this embodiment, the pharmaceutical composition comprises by weight 3 to 5% of realgar, 27 to 35% of indigo naturalis, 52 to 57% of salvia miltiorrhiza, and 7 to 9% of heterophylla.

Preferably, the indigo naturalis is excellent grade indigo naturalis or special grade indigo naturalis. Based on the existing methods, the indigo naturalis is more preferably the special grade indigo naturalis, and the special grade indigo naturalis is formed after being cleaned, refined, and sterilized to increase the indirubin content. The excellent grade indigo naturalis and special grade indigo naturalis have the following characteristics: the excellent grade indigo naturalis and the special grade indigo naturalis have a water content of not higher than 6.0 wt. %; and 10 g of excellent grade indigo naturalis or special grade indigo naturalis is dissolved in 100 mL water and is stirred for 25 min, and the resulting mixture has a pH of 7.0-9.0.

The present disclosure also provides a method for preparing a pharmaceutical composition for treating leukemia, including the following steps:
a) mixing a realgar, a heterophylla and an indigo naturalis powder uniformly to obtain a first mixture;
b) adding water to salvia miltiorrhiza at a ratio of 8 to 9 mL of water per 1 g of salvia miltiorrhiza to obtain a second mixture, decocting the second mixture 1-2 times, and for 50-55 min each time, then filtering the resultant mixture to obtain a filtrate, then concentrating the filtrate at a temperature of 48-54° C. to yield a concentrate; and
c) mixing the first mixture and the concentrate, and forming the resultant mixture into a pharmaceutical composition in a dosage form.

In a class of this embodiment, the realgar is prepared as follows: mixing 1 part by weight of orpiment, calcite, quartz, or gypsum with 0.5 part by weight of water, grinding the mixture for 5-10 minutes to yield a paste, adding 40 parts by weight of water to the paste and stirring for 2 minutes, allowing the mixture to stand still for 5 minutes, separating the suspension and the precipitate and collecting the suspension, then grinding the precipitate; repeating the operations of allowing the ground mixture to stand still, separating the suspension and the precipitate, and grinding the precipitate; combining the collected suspensions and allowing it to stand for 12 hours or more. The supernatant is poured off, and the solid is filtered and dried at 60° C. for 10 hours or less.

In a class of this embodiment, the indigo naturalis is prepared as follows: collecting stalks and leaves of a plant selected from Baphicacanthus cusia (Nees) Bremek., Polygonum tinctorium Ait., and Isatis indigotica Fort; cutting the stalks into pieces, soaking 1 part by weight of the pieces and the leaves in 30 parts of water for 3-5 days until the stalk pieces peel, the leaves decay, and the color of the supernatant becomes dark green; removing the residues of the stalks and leaves, filtering the supernatant by a 40 mesh sieve, adding 5 kg calcium oxide to 50 kg of the filtrate, stirring and allowing the mixture to stand still until the mixture turns from dark green to purple, discarding the supernatant, adding 500 kg water, stirring and collecting the foam on the liquid surface, drying the foam, pulverizing the dried foam and sieving with a 100 mesh sieve to obtain the indigo naturalis.

In a class of this embodiment, the heterophylla is prepared as follows: removing roots from the plant of heterophylla, soaking the heterophylla in boiling water, drying and pulverizing the heterophylla to powders.

The heterophylla is gradually broken to form powder. Specifically, the heterophylla is pulverized into a fine powder; then, the fine powder is crushed to a very fine powder for later use.

The fine powder refers to powder that can completely pass through the No. 6 sieve and no less than 95% can pass through the No. 7 sieve; the very fine powder refers to powder that can completely pass through the No. 8 sieve and no less than 95% can pass the No. 9 sieve.

Further, the pharmaceutical composition is in a dosage form of tablets, pills, capsules, or granules.

In a class of this embodiment, the concentration of the filtrate is carried out as follows: disposing the filtrate in a concentrator having an absolute pressure of smaller than −0.08 MPa, and concentrating at a vapor pressure of not higher than 0.25 MPa and a temperature of 48-54° C. to yield a paste having a relative density of 1.15 to 1.24 at 50° C. with respect to water (the same below).

Specifically, the pharmaceutical composition is in the dosage form of tablets; the concentrate of b) is a clear paste with a relative density of 1.15 to 1.20 measured at a temperature of 50° C.; and then the clear paste is mixed with the first mixture of a), granulated, dried, sized, compressed, and coated to obtain the tablets.

Further the pharmaceutical composition is in the dosage form of pills; the concentrate of b) is concentrated to a thick paste, then the thick paste is dried under reduced pressure into a dry paste, the dry paste is crushed into a fine powder to obtain a dry fine powder; the dry fine powder is mixed with the first mixture of a), and placed in a pill making machine with water and then dried to obtain the pills.

Further the pharmaceutical composition is in the dosage form of capsules; the concentrate of b) is a clear paste with a relative density of 1.15 to 1.20 measured at a temperature of 50° C.; and then the clear paste is mixed with the first mixture of a), granulated, dried, sized, compressed, and filled into capsules.

The pharmaceutical composition for treating leukemia provided by the present disclosure is mainly clinically used for acute promyelocytic leukemia or chronic myelogenous leukemia.

Compared with the prior art, the beneficial effects of the present disclosure are as follows:

1. The pharmaceutical composition for treating leukemia provided by the present disclosure increases the amount of salvia miltiorrhiza from 36%-46% to 50%-60%, and reduces the amount of realgar from 12%-18% to 2%-8%. Clinical trials have surprisingly found that pharmaceutical compositions modified according to the prior art are clinically used for the treatment of leukemia, and are mainly used for acute promyelocytic leukemia and chronic myelogenous leukemia, the curative effect has been significantly improved, and the toxic and side effects have been greatly reduced.

2. The active ingredients of salvia miltiorrhiza, such as tanshinone and salvianolic acid, are unstable under long-term high-heat conditions. The extraction time in the prior art is 1-2 hours. Under these conditions, most of the active ingredients of salvia miltiorrhiza have been destroyed. In order to ensure that the active ingredients of salvia miltiorrhiza are not destroyed, there is a strict requirement on the extraction time of salvia miltiorrhiza. In the preparation method of pharmaceutical composition for treating leukemia provided by the present disclosure, the amount of water added to salvia miltiorrhiza is reduced, the number of decocting times is reduced, and the decocting time is shortened to ensure that the active ingredients of salvia miltiorrhiza are not damaged. The experimental results show that the active ingredients of salvia miltiorrhiza are tanshinone IIA (C19H18O3), tanshinone I (C18H12O3), and tanshinone IIB (C19H18O4), etc., all of which can be well extracted. Tanshinone IIA, the active ingredient as the control index component, is detected by high performance liquid chromatography and the extraction rate of tanshinone HA reaches 75%. The activity of each active ingredient is well maintained and the extraction rate of the active ingredient from the raw materials is maintained at a high level.

3. The pharmaceutical composition for treating leukemia provided by the present disclosure is suitable for dosage forms including but not limited to tablets, capsules, granules, pills and the like.

### DETAILED DESCRIPTION OF THE EMBODIMENTS

The implementations of the present disclosure will be described in detail with the following embodiments, but it will be understood by those skilled in the art that the following embodiments are only illustrative of the present disclosure and should not be construed as limiting the scope of the present disclosure. If no specific conditions are indicated in the examples, it shall be performed according to conventional conditions or conditions recommended by the manufacturer. The reagents or instruments used which are not specified by the manufacturer are all commercially available conventional products.

In this disclosure, the active components of the pharmaceutical composition are prepared as follows:

Realgar was extracted as follows: the realgar without impurities was taken and mixed with water (a mixed ratio of water to material was 0.5 mL/per gram). The mixture was ground for 5-10 min to be pasty, stirred with water (a mixed ratio of water to material was 40 mL/per gram) for 2 min, allowed to stand for 5 min. The supernatant was discarded, and the precipitate was collected and ground. Repeat the above operations. The suspension was combined, allowed to stand for 12 h or more. The supernatant was discarded. A solid was collected, filtered, and dried at 60° C. for 10 h or less, to yield realgar.

Indigo naturalis was extracted as follows: the leaves and the stems of Baphicacanthus cusia (Nees) Bremek. were cut into small pieces, and water (a mixed ratio of water to material was 30 mL/per gram) was added and soaked for 3-5 days until the stems were peeled and the leaves were rotted. When the extract was dark green, the stems and leaves were removed. The obtained extract was screened using a 40-mesh sieve, and 5 kg of lime (CaO) per 50 kg of the medicinal material was mixed with the extract. The mixture was stirred and allowed to stand. When the extract was changed from blackish green to purple, the supernatant was discarded, and water and the medicinal material was mixed (a mixed ratio of water to material was 10 mL/per gram), stirred, and a foam on the liquid surface was collected, dried, pulverized, and screened using a 100-mesh sieve, to yield indigo naturalis.

Salvia miltiorrhiza was extracted as follows: the impurities of the pure root of red-rooted salvia was removed. The pure root was cut, and dried to obtain the pure medicinal material. The medicinal material was mixed with water (a mixed ratio of water to material was 8-9 mL/per gram), boiled and extracted for 1-2 times, each for 50-55 min. The filtrate was combined and concentrated to obtain a clear paste with a relative density of 1.15-1.24 (50° C.).

Heterophylla was extracted as follows: the medicinal herbs of Radix Pseudostellariae without fibrous roots was boiled in boiling water, dried, pulverized into fine powders in a pulverizer, and further pulverized into ultrafine powders in an ultrafine pulverizer.

### Example 1

Realgar, indigo naturalis, salvia miltiorrhiza, and heterophylla were prepared with a weight percentage of 2%, 42%, 50% and 6%, respectively.

Water was added to the salvia miltiorrhiza at a ratio of 8 mL of water per 1 g of the salvia miltiorrhiza, decocted 2 times, for 50 min each time, then filtered to obtain a filtrate, the filtrate was then combined and concentrated at a temperature of 48-54° C. to yield a clear paste with a relative density of 1.15 to 1.20 (50° C.).

The homogenized fine powders of realgar, indigo naturalis, and heterophylla were added to the clear paste, granulated, dried, sized, and compressed (weight 0.25 g), and coated to obtain the pharmaceutical composition for treating leukemia.

### Example 2

Realgar, indigo naturalis, salvia miltiorrhiza, and heterophylla were prepared with a weight percentage of 8%, 25%, 60% and 7%, respectively.

Water was added to the salvia miltiorrhiza at a ratio of 9 mL of water per 1 g of the salvia miltiorrhiza, decocted 2 times, for 55 min each time, then filtered to obtain a filtrate, the filtrate was then combined and concentrated at a temperature of 48-54° C. to yield a clear paste with a relative density of 1.15 to 1.20 (50° C.).

The homogenized fine powders of realgar, indigo naturalis, and heterophylla were added to the clear paste, granulated, dried, sized, and compressed (weight 0.25 g), and coated to obtain the pharmaceutical composition for treating leukemia.

### Example 3

Realgar, indigo naturalis, salvia miltiorrhiza, and heterophylla were prepared with a weight percentage of 5%, 27%, 58% and 10%, respectively.

Water was added to the salvia miltiorrhiza at a ratio of 8 mL of water per 1 g of the salvia miltiorrhiza, decocted 2 times, for 55 min. each time, then filtered to obtain a filtrate, the filtrate was then combined and concentrated to yield a thick paste, then the thick paste is dried and crushed into a fine powder.

The fine powder of salvia miltiorrhiza were added into the homogenized fine powders of realgar, indigo naturalis, and heterophylla, mixed uniformly, placed in a pill making machine, panned with water and dried to obtain pills with a weight of 0.25 g.

### Example 4

Realgar, indigo naturalis, salvia miltiorrhiza, and heterophylla were prepared with a weight percentage of 4%, 35%, 52% and 9%, respectively.

Water was added to the salvia miltiorrhiza at a ratio of 9 mL of water per 1 g of the salvia miltiorrhiza, decocted 2 times, for 50 min each time, then filtered to obtain a filtrate, the filtrate was then combined and concentrated at a temperature of 48-54° C. to yield a clear paste with a relative density of 1.15 to 1.20 (50° C.).

The homogenized fine powders of realgar, indigo naturalis, and heterophylla were added to the clear paste, granulated, dried, sized, and filled into a capsule (the weight is 0.25 g) to obtain the pharmaceutical composition for treating leukemia in a capsule form.

### Example 5

Realgar, indigo naturalis, salvia miltiorrhiza, and heterophylla were prepared with a weight percentage of 3%, 32%, 57% and 8%, respectively.

Water was added to the salvia miltiorrhiza at a ratio of 9 mL of water per 1 g of the salvia miltiorrhiza, decocted 2 times, for 55 min each time, then filtered to obtain a filtrate, the filtrate was then combined and concentrated at a temperature of 48-54° C. to yield a clear paste with a relative density of 1.15 to 1.20 (50° C.).

The homogenized fine powders of realgar, indigo naturalis, and heterophylla were added to the clear paste, granulated, dried, sized, and filled into a capsule (weight 0.25 g) to obtain the pharmaceutical composition for treating leukemia in a capsule form.

### Example 6

Different dosage forms of the pharmaceutical compositions prepared in Examples 1-5 were used in clinical validation for treating leukemia. The compound realgar natural indigo tablet prepared by the example of Chinese Patent Application No. 200410050453.1 was used as the control group. A total of 600 patients were selected and randomly divided into six groups, that is, 100 patients in each group. On the first day of treatment, 3 to 5 tablets or pills or capsules were taken each time, 3 times a day, and on and after the 10th days, 30 tablets or pills or capsules were taken per day, and 10 for each time. The control group was taken according to the administration method taught in Chinese Patent Application No. 200410050453.1.

Efficacy criteria (cf. Suzhou National Leukemia Chemotherapy Symposium, 1987):

Complete Remission (CR): No clinical symptoms or signs due to leukemia cell infiltration, normal or nearly normal life, hemogram: Hb≥100 g/L (male) or ≥90 g/L (female), neutrophil absolute value ≥1.5×10⁹/L, platelet ≥100×10⁹/L, peripheral blood classification without leukemia cells, myelogram: the myeloblast+promyelocyte −5%, red blood cells and megakaryocytes are normal.

Partial remission (PR): myeloblasts of bone marrow+promyelocytes of bone marrow >5% and myeloblasts of bone marrow+promyelocytes of bone marrow ≤20%, or one of the clinical and hemogram did not reach the standard of complete remission.

Complete remission rate=numbers of complete remission per group/total numbers of patient per group; partial remission rate=1−complete remission rate. The specific results are shown in Table 1.

TABLE 1
Comparison of efficacy of pharmaceutical compositions
of the disclosure and control group

| Groups | Complete remission rate (%) | Partial remission rate (%) |
|---|---|---|
| Example 1 | 92.9% | 7.1% |
| Example 2 | 93.0% | 7% |
| Example 3 | 93.8% | 6.2% |
| Example 4 | 94.1% | 5.9% |
| Example 5 | 93.6% | 6.4% |
| Control Group | 82.1% | 17.9% |

As shown in Table 1, the therapeutic effect of the pharmaceutical compositions for treating leukemia provided by the present disclosure is higher than that of the control group.

Unless otherwise indicated the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A pharmaceutical composition for treating leukemia, the composition comprising by weight 2 to 8% of realgar, 25 to 42% of indigo naturalis, 50 to 60% of salvia miltiorrhiza, and 6 to 10% of heterophylla.

2. The pharmaceutical composition of claim 1, wherein the pharmaceutical composition comprises by weight 3 to 5% of realgar, 27 to 35% of indigo naturalis, 52 to 57% of salvia miltiorrhiza, and 7 to 9% of heterophylla.

3. The pharmaceutical composition of claim 1, wherein the indigo naturalis is excellent grade indigo naturalis or special grade indigo naturalis.

4. A method for preparing the pharmaceutical composition of claim 1, the method comprising:
mixing a realgar powder, a heterophylla powder, and an indigo naturalis powder to obtain a first mixture;
adding water to the salvia miltiorrhiza at a ratio of 8-9 mL of water per 1 g of the salvia miltiorrhiza to obtain a second mixture, decocting the second mixture 1-2 times, for 50-55 min each time, then filtering to obtain a filtrate, and concentrating the filtrate at a temperature of 48-54° C. to yield a concentrate; and
mixing the first mixture and the concentrate to form a pharmaceutical composition.

5. The method of claim 4, wherein the pharmaceutical composition is in a dosage form of a tablet, a pill, a capsule, or a granule.

6. The method of claim 5, wherein the pharmaceutical composition is in the dosage form of a tablet; the concentrate is a clear paste with a relative density of 1.15 to 1.20 measured at a temperature of 50° C.; and the clear paste is mixed with the first mixture, granulated, dried, sized, compressed, and coated to obtain the tablet.

7. The method of claim 5, wherein the pharmaceutical composition is in the dosage form of a pill; the concentrate is concentrated to a thick paste, then the thick paste is dried under reduced pressure into a dry paste, the dry paste is crushed into a fine powder to obtain a dry fine powder; and the dry fine powder is mixed with the mixture, and placed in a pill making machine with water and then dried to obtain the pill.

8. The method of claim 5, wherein the pharmaceutical composition is in the dosage form of a capsule; the concentrate is a clear paste with a relative density of 1.15 to 1.20 measured at a temperature of 50° C.; and the clear paste is mixed with the mixture, granulated, dried, sized, compressed, and filled into capsule.

9. The pharmaceutical composition of claim 2, wherein the indigo naturalis is excellent grade indigo naturalis or special grade indigo naturalis.

10. A method for preparing the pharmaceutical composition of claim 2, the method comprising:
mixing a realgar powder, a heterophylla powder, and an indigo naturalis powder to obtain a first mixture;
adding 8-9 times amount by weight of water to salvia miltiorrhiza to obtain a second mixture, decocting the second mixture 1-2 times, for 50-55 min each time, then filtering to obtain a filtrate, concentrating the filtrate at a temperature of 48-54° C. to yield a concentrate; and
mixing the first mixture and the concentrate to form a pharmaceutical composition in a dosage form.
